(19) United States
(12) Patent Application Publication
(10) Pub. No.: US 2019/0338341 A1
(43) Pub. Date: Nov. 7, 2019

(54) Methods and device for electromagnetic detection of polymerase chain reaction

(71) Applicant: Vladimir Gusiatnikov, San Francisco, CA (US)

(72) Inventor: Vladimir Gusiatnikov, San Francisco, CA (US)

(73) Assignee: Molde, San Francisco, CA (US)

(21) Appl. No.: 16/403,534

(22) Filed: May 4, 2019

Related U.S. Application Data
(60) Provisional application No. 62/667,421, filed on May 5, 2018.

(51) Int. Cl. C12Q 1/686; G01N 15/10

(52) U.S. Cl. CPC C12Q 1/686; G01N 2015/0065; G01N 15/1031

(57) ABSTRACT

In a sample—reagent mix for polymerase chain reaction (PCR), forward primers are attached to superparamagnetic beads. During a part of a cycle of PCR, beads with bound amplicons are attracted to an electromagnet coil. Electrodeless, wireless detection of PCR and nucleic acid quantitation are achieved in real time by placing the beads in the ac electromagnetic field of the same, and/or another, coil and measuring, with one or both coils, cycle-by-cycle changes in the electrical conductivity and/or the complex permittivity of the aggregate of beads, amplicons, and the interspersed reaction mix. After the cycle's measurement is complete, the beads are redispersed within the reaction mix by coordinated action of the first coil and a third coil.

### REFERENCES

[1] D. Dressman et al., Proc. Natl. Acad. Sci. U.S.A. 100, 8817 (2003).

[2] QIAGEN N.V., DNeasy® Blood & Tissue Handbook (Venlo, The Netherlands, 2006), p. 23.

[3] S. Tomić et al., Phys. Rev. E 75, 021905 (2007).

[4] Promega Corp., Protocols & Applications Guide, rev. 12/09 (Madison, Wis., 2009), p. 1-12.

[5] H. Ma et al., Sci. Rep. 3, 2730 (2013).

[6] Y. Cai et al., BioMed Res. Int. 2014, 810209 (2014).

[7] E. Gheorghiu et al., U.S. Pat. No. 9,315,855 (19 Apr. 2016).

[8] M. Nakano, Z. Ding, and J. Suehiro, Biosensors 7, 44 (2017).

[9] M. Nakano, Z. Ding, and J. Suehiro, Microfluid. Nanofluidics 22, 26 (2018).

[10] V. Gusiatnikov, U.S. patent application Ser. No. 16/288,365 (28 Feb. 2019, unpublished).

### FIELD

The proposed classification of this patent is C120 2563/116, GO1N 27/023.

The invention relates to detection of nucleic acid amplification products in the course of polymerase chain reaction by measuring their electrical properties. The invention relates to determining the starting quantity (quantitation) of a nucleic acid template using kinetic amplification curves (real-time, or quantitative, polymerase chain reaction). More particularly, the invention relates to the detection and quantitation involving measurement of the electrical conductivity and/or the complex permittivity of a reaction mix extract containing the amplified nucleic acids.

From a complementary angle, the invention relates to the measurement of the electrical conductivity and/or the complex permittivity of a settled suspension layer, wherein said layer contains nucleic acid amplification products. Most specifically, the invention relates to non-contact electromagnetic measurement of the electrical conductivity and/or the complex permittivity of a settled suspension layer where the layer is placed in the electromagnetic field of a coil. The invention further relates to electrical measurement involving synchronous detection, in particular phase-sensitive detection.

### BACKGROUND

The invention derives upon the method and devices for wireless detection of nucleic acid amplification disclosed in Patent Application [10] and describes an additional distinct method for such detection. The present invention further specifies the means for electromagnetically extracting specific amplification products into a settled suspension layer, thereby concentrating the products approximately thousandfold. Unlike the invention of Patent Application [10], the present invention is limited to a single amplification scheme, that of qPCR (real-time, or quantitative, PCR).

As currently practiced, real-time detection of PCR overwhelmingly uses fluorescence. Light from a source excites a fluorophore within a reaction mix whose activity depends on the quantity of amplicons. The fluorophore emits light at a wavelength different from that of the incident light. The separation of wavelengths required for fluorescent detection necessitates optical filtering. These filters, excitation sources, and optical detectors add cost, weight, size, and complexity, precluding the development of affordable, small, rugged, robust qPCR systems. Another contributor to the weight, size, and cost is the requirement to condensation-proof the optical path; both water evaporated from the sample and water absorbed in the instrument from the ambient can condense on the optical components as the temperature is reduced well below its denaturation-step value for the subsequent annealing step, thereby altering the transmission of light.

Among alternative methods for real-time PCR detection and quantitation, purely electrical ones will serve to minimize the weight, cost, and size of the resulting instrument. However, contact measurement of the electrical properties of the reaction mix carries complications and is therefore not widely accepted in the art. Foremost, electrodes contacting the reaction mix are chemically active and can be fouled. While amplification assays are engineered with the chemical activity in mind, certain sample matrices are incompatible with certain electrode materials, and unexpected sample ingredients can alter the reaction in unpredictable ways. Even a minimally reactive electrode is still subject to physical fouling by, and to surface degradation from, sample contaminants. Signal change caused by slow oxidation or aggregation on the surface of the electrode can be indistinguishable from a kinetic curve. Contact electrical measurement is therefore best suited for the detection of purified nucleic acids and not to real-life biological specimens.

Electrodes can be passivated with an oxide layer or separated from the mix by a dielectric. Non-contact measurement involving electrodes and its subset, contactless capacitively coupled conductivity detection, are known in the art. However, it is difficult to insert sufficient electric field across the capacitive barrier and into the mix to probe, with acceptable signal-to-noise, the electrical properties without employing frequencies in the tens of megahertz, which necessitate complex electronics. Adding an inductor to the circuit can partially null the effects of the capacitance of the separation barrier.

For both contact and contactless techniques, electrodes incur a cost to engineer and manufacture. In an instrument comprising a disposable cartridge and a permanent reader, a set of electrical connections must be engineered between the cartridge and the reader. Per unit of mass, connectors are the most expensive class of electronic components. Additionally, electrodes must be provided within each single-use cartridge if the technique used is contact. Micro- or nanolithography for electrode patterning is the most expensive process step of cartridge manufacturing.

Another problem with qPCR detection as currently practiced and with many of the alternative detection methods, including embodiments of the method disclosed in Patent Application [10], is the use of a toxic dye. Non-specific fluorescent detection commonly employs DNA-intercalating dyes as probes. Electrochemical detection methods use an intercalating agent to achieve a lower limit of quantitation comparable with that of fluorescent detection, and wireless detection as previously invented by the applicant cannot be made sufficiently sensitive without the use of an intercalating probe if the amplification scheme is qPCR. An agent that mechanically alters the structure of DNA is by definition mutagenic to humans. Although newer dyes are claimed to be less toxic than first-generation ethidium bromide and the quantities of the dye in each disposable cartridge are too small to cause measurable health effects in case of accidental operator exposure or ingestion, repeated exposure of manufacturing workers and researchers to the dyes may pose a problem. Insufficient epidemiological data exists concerning long-term safety of the newer dyes.

Other nucleic acid amplification schemes known in the art include loop-mediated isothermal amplification (LAMP), strand displacement amplification (SDA), recombinase polymerase amplification (RPA), helicase-dependent amplification (HDA), multiple-displacement amplification (MDA), rolling-circle amplification (RCA), and nucleic acid sequence-based amplification (NASBA). Electrical detection has been applied to all of these schemes. In particular, LAMP can be detected by purely electrical means with sufficient sensitivity and without the use of a probe owing to the formation in its course of an insoluble molecule. Ensuingly, LAMP lends itself well to detection with the wireless method disclosed in Patent Application [10].

However, PCR in general and qPCR in particular have behind them by far the largest body of expertise, accumulated over the course of more than 30 years and more than 15 years, respectively. Optimized, fully debugged primer sets are readily available in the public domain for most common target pathogens, and PCR reagent mixes have been produced fitting all common sample kinds and matrices. By comparison, there is a relative lack of awareness and acceptance of alternative amplification schemes in the research community. Amending qPCR with a modern detection method is a path to quickly develop and validate an affordable, small, rugged, and robust molecular diagnostic or molecular detection system—at the expense of retaining the complex, power-hungry thermal cycling regime of PCR. Care must be exercised in the design to maintain the sensitivity and the specificity that PCR allows.

As disclosed in Patent Application [10], wireless detection of amplification monitors, in real time, bulk electrical properties of a mix containing reaction products. The electrical conductivity of a PCR mix is not dramatically altered by the incorporation of dNTPs into strands at DNA concentrations below the limit at which the activity of polymerase is inhibited. Said limit is approximately 1.0×10⁻⁵ by weight (10 ng/μl) [4, 8] and is independent of the strand length [4]; amplification will saturate at this concentration and additional cycles will not increase the amplicon quantity. The conductivity of a fluid containing a PCR-limiting quantity of amplicons is not much different from that of a fluid containing a smaller amount, and is primarily determined by the concentration of ionic salts. The real part of the complex permittivity shows more change owing to polarization effects; the dipole behavior of the DNA double helix is materially different from that of dNTPs in solution. However, concentrations of double-stranded DNA in a fluid mix higher than the amplification-saturating limit yield [3, 5, 8] far more contrast in both in-phase and out-of-phase electrical response; for example, going from 10⁻⁴ to 10_² is strongly preferred to comparing 10⁻⁷ and 10⁻⁵. It would therefore be quite desired to extract and concentrate the amplicons prior to measuring their electrical properties.

Such approach is indeed taken by some of the work in the art [5, 8, 9] but endpoint detection employed therein inherently limits the dynamic range of quantitation. Another challenge to quantitation is posed by the presence of overwhelming amounts of native DNA. In many real-life sample kinds, the quantity of the nucleic acid of the pathogen under examination is orders of magnitude less than the quantity of the DNA of the species the sample came from, both in copy number and by weight. If the concentration of native DNA in the sample—reagent mix exceeds the amplification saturation limit, the mix must be diluted prior to amplification. Doing so increases (worsens) the lower limit of pathogen detection.

If the dilution is insufficiently aggressive and the method of detection is not specific to the nucleic acid target sequence of interest, there will not be a lot of detection contrast. The achievement of a quantitation threshold may therefore be masked by noise, and the accuracy of the quantitation will suffer. For example, DNA content of raw chicken breast is approximately 3.6×10⁻⁵ by weight [6]. This number is also within the general range of total DNA content of mammalian blood [2]. Finding a small number of copies of a common food pathogen bacterium within ground chicken breast, or a small number of viral copies within DNA extracted from blood, requires that the sample be diluted to below 1.0×10⁻⁵ DNA by weight.

Suppose the ground-chicken sample is diluted tenfold, increasing the lower limit of detection by the same factor. If the method detects all DNA and not the specific pathogen DNA, the detected nucleic acid concentration at the beginning of the reaction, corresponding to near-zero amplicons, will be 3.6×10⁻⁶ and the maximum, amplification-limiting DNA concentration will reach 1.0×10⁻⁵ after a number of PCR cycles—a detection dynamic range of less than 3×; clearly susceptible to noise caused by concentration fluctuations and therefore suboptimal if the detection response is roughly linear, and catastrophic if logarithmic. A higher dilution will improve the quantitation accuracy at the expense of increasing the lower limit of detection.

A specific detection method is therefore preferred. If the detection is specific, the sample need not be diluted considerably beyond the polymerase activity inhibition limit.

### SUMMARY

Polymerase chain reaction is performed as described in the art and as modified below. Methods and a device for electromagnetic detection of the reaction's products disclosed herein employ a hold coil, an excitation coil, a pickup coil, and a release coil. These functions are realized with two or three distinct coils, none of which are in direct contact with the reaction mix.

In some embodiments of the methods, forward primers are attached to superparamagnetic beads prior to the beginning of the reaction. Alternatively, the reaction begins with free primers whose 5′ ends are conjugated with the moiety necessary to subsequently attach to the beads. After a number of reaction cycles, some of the primers are extended to moiety-terminated double-stranded amplicons whereas the remainder of the primers remain unextended in the mix. The reaction mix is then brought into contact with the beads and both the unextended primers and the amplicons attach to the beads. The chain reaction proceeds thereon with the beads included in the reaction mix.

After, or shortly before, the completion of the elongation step of a cycle of the reaction, a magnetic field gradient attracts the beads to the vicinity of the hold coil, thereby concentrating the amplicons. Said magnetic field is produced by supplying an electric current to the hold coil; said current may be dc or another waveform. Upon sufficient settling of the bead-amplicon condensate, its electrical conductivity and/or complex permittivity are interrogated with the excitation coil and the pickup coil, which in some device embodiments are the same coil.

Three distinct embodiments of the electromagnetic detection method are disclosed in the appended claims. The first two embodiments add cycle-by-cycle magnetic-bead amplicon extraction and redispersion steps to embodiments of the method disclosed in Patent Application [10], and the third embodiment has heretofore not been disclosed.

In all embodiments of the method, an ac voltage is applied to the excitation coil, creating an ac electromagnetic field within the bead-amplicon condensate. The electrical conductivity and the complex permittivity of the condensate are determined, in part, by the quantity of the amplicons. Their accumulation will alter said properties, and this effect is sensed by monitoring the voltage across and/or the current through the excitation coil, the pickup coil, or both. In an embodiment of the method, the pickup coil is driven at or near resonance and the excitation frequency is continuously adjusted using a measured signal amplitude so as to optimize (that is, to maximize or to minimize) the signal.

In an embodiment of the method, the excitation coil's field is sufficient to magnetically saturate the beads during a portion of the period of the excitation. The varying effective permeability of the bead-amplicon condensate alters the effective lumped inductance of the pickup coil. Said inductance is measured by applying a second ac voltage at a second frequency to the pickup coil, said frequency being at or near the resonance frequency of the pickup coil's circuit when the beads are in saturation. The effective inductance is then demodulated against the first excitation frequency. Its phase shift with respect to the first excitation relates to the electrical conductivity and the complex permittivity of the condensate.

Quantitation is achieved by measuring the cycle number (which need not be an integer) required for a function of the following parameters: the amplitudes and/or the phases of the monitored voltages and/or currents; and/or of the excitation frequency; or instead, in some embodiments, of the effective lumped inductance of the pickup coil and/or its phase relative to the original excitation voltage; to reach a predetermined threshold.

In order to resume the chain reaction, beads with the attached amplicon are redispersed upon completion of the cycle's measurement and the chain reaction proceeds with the next cycle's denaturation step. Coordinated electric current waveforms are supplied to the hold coil and to the release coil. The field of the latter attracts the beads away from the hold coil and into the reaction mix. Repeated agitation serves to stir and disperse the beads as uniformly as practically possible within the mix, reducing the amplicon concentration within the mix to that below the critical limit at which the activity of polymerase is inhibited.

Further, a device utilizing the electromagnetic detection method and disclosed herein comprises two or three coils. The first coil is a helical coil wound around a core of a high permeability and serves as the hold coil. The second coil is a planar (spiral) coil disposed on the opposite side of the sample space; this coil is the release coil. In embodiments of the device, the hold coil serves as the excitation coil and a third coil which is a planar (spiral) coil disposed in the proximity of the reaction mix serves as the pickup coil. In embodiments of the device, the third coil, driven at or near resonance, serves as both the excitation coil and the pickup coil. In embodiments of the device, the third coil is not present and the hold coil, driven at or near resonance, is both the excitation coil and the pickup coil. In embodiments of the device, the hold coil is the excitation coil that drives the beads into magnetic saturation and the separate planar (spiral) pickup coil exhibits resonance for a portion of the excitation period.

In some device embodiments, a fluxmat abutting the release coil serves to increase the magnitude of the magnetic field gradient to which the beads are exposed upon application of a current through the coil. Finally, multiple instances of the device herein disclosed may be assembled into an instrument.

Adding cycle-by-cycle magnetic-bead extraction to wireless amplification detection enables the method to retain the high sensitivity and specificity that are achievable with the gold-standard qPCR with fluorescent detection.

### PRIOR ART

The use of magnetic beads and magnets in general to extract, hold, move, and manipulate nucleic acid molecules is widely known in the art. Combined with the use of electromagnets, said techniques are less common.

The applicant is unaware of methods or devices for real-time detection of products of polymerase chain reaction combining, in a single technique or apparatus, cycle-by-cycle extraction of amplicons with the aid of magnetic beads on the one hand; and non-contact measurement in general, by purely electrical means, of the quantity of the amplicons on the other hand. In particular, electrodeless detection of nucleic acid amplification reaction products involving one or more coils is not known to the applicant to have been invented, conceived, or reduced to practice, whether combined with a method for concentrating said products or not.

However, contact [7, 8, 9] and non-contact [7] electrical measurement of nucleic acid amplification reaction products following endpoint magnetic-bead extraction and involving electrodes is known in the art. From a complementary angle, electrodeless measurement of the electrical conductivity of an object in general, and a fluid analyte in particular, that utilizes electromagnetic fields generated by, and/or detected by, coil(s) is widely practiced in various fields outside of the primary field of the invention

### APPLICATION

The methods and device disclosed herein enable specific detection of PCR amplicons, and the determination of the starting quantity of a nucleic acid template in a sample, via non-optical, non-contact, robust, and sensitive means. The excitation and the readout are completely electrical, eliminating electrooptical and optical components in the resulting instrument. Contactless detection provides for a robust amplification reaction free of interference from the interaction between the electrode material and the sample matrix, and for the unambiguous interpretation of its results, unsullied by the interaction of the electrodes with sample contaminants. The lack of electrodes further eliminates electrical connectors; all electrical parts are kept out of the sample space, allowing for a simple reader—cartridge interface and a straightforward cartridge manufacturing process.

Magnetic-bead extraction in the course of a PCR cycle concentrates the amplicons approximately thousandfold without the use of permanent magnets or moving parts, thereby increasing the sensitivity of the detection. The method isolates and detects specific amplification products within a small volume while nonspecific nucleic acid strands remain dispersed in the reaction mix where they do not materially affect the detection; the separation endows the quantitation with specificity. Subsequent redispersion of the beads allows for real-time, cycle-by-cycle detection, endowing the quantitation with a large dynamic range. Finally, the method does not employ potentially mutagenic intercalating agents, reducing the level of health risk in manufacturing and in end use.

The described method of electromagnetic detection thereby enables safe, affordable, small, rugged, robust, sensitive, and specific instruments and devices for molecular detection and molecular diagnostics.

### BRIEF DESCRIPTION OF THE DRAWINGS

While particular embodiments of a device have herein been described and illustrated to exemplify the principle of the invention, such description is not intended to be limiting. Modifications, changes, adaptations, and juxtapositions may become apparent to those skilled in the art, and it is intended that the invention be limited only by the scope of the appended claims. For example, if a specific arrangement of coils and a particular device embodiment are illustrated in a drawing, the description is not restricted to limit the use of said type of coil arrangement exclusively with the particular device kind.

FIG. 1 is a perspective view of a device for electromagnetic detection and quantitation of PCR products. Said device comprises three coils situated in the proximity of a sample well, and the drawing depicts their arrangement.

FIG. 2 is a perspective view of such device comprising a reduced number of coils.

FIG. 3 is a schematic view of a functionalized superparamagnetic bead that is an aspect of the invention.

### DETAILED DESCRIPTION OF THE METHODS

Described in Sections 10 through 12 are methods and a device for electromagnetic detection of polymerase chain reaction. Compared with the art, the three steps of: denaturation, annealing, and elongation are amended with a fourth step comprising the measurement of the quantity of amplicons. Said amplicons are concentrated with electromagnetic means for the purpose of said measurement at the beginning of the step, and redispersed with electromagnetic means upon completion of the measurement. Certain embodiments of the methods amend the method for wireless, or electrodeless, detection of nucleic acid amplification disclosed in Patent Application [10]. Another method embodiment disclosed herein is new. While a detailed description of the methods and their embodiments serves to explain the principles of the invention, such description is not intended to be limiting. Modifications, equivalents, adaptations, and alternatives may become apparent to those skilled in the art, and it is intended that the invention be limited only by the spirit and the scope of the appended claims.

The invention applies handling and interrogation of nucleic acid material with coils, and the technique of phase-sensitive detection, to real-time determination, in the course of PCR, of the starting quantity of a nucleic acid template present in a sample. The sample is brought into contact with a mix containing, in sufficient quantity, all reagents necessary for an amplification reaction to occur, including a catalyzing polymerase and possibly other enzymes, nuclease-free water, primers designed to amplify the specific target sequence, dNTPs, magnesium chloride and potassium chloride, and reaction buffers. In embodiments of the methods, some or all of the forward primers are attached to superparamagnetic beads prior to the first reaction cycle by means known in the art.

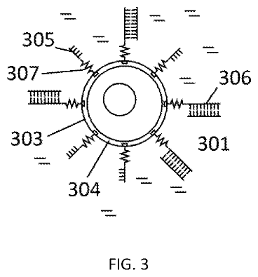

In other embodiments, conditions are created for a nucleic acid sequence in the template to undergo the amplification and the reaction proceeds for a number of cycles before the beads are introduced into the reaction mix. In the latter case, some or all of the forward primers are conjugated with a moiety, such as biotin, prior to the reaction. The beads are coated with a complementary binding moiety, such as streptavidin and where the bound complex possesses a high resistance to solvents, detergents, and extremes of temperature. A modern avidin such as Tamavidin® 2 or Tamavidin® 2-HOT possesses a higher resistance to the temperature required during the denaturation step than streptavidin and is used in an embodiment. Amplicons generated as product of the initial reaction cycles will carry the first moiety as a result of forward primer extension and will therefore also bind to the beads as illustrated in FIG. 3.

Following the binding of the beads to the forward primers and, in some embodiments, to the amplicons, the resulting reaction mix is situated in a sample space such as a well or a channel and conditions are created for amplification as described in the art; the temperature of the mix is cycled between values required for the denaturation step, the annealing step, and, if a set temperature is necessary for such, the elongation step. Said conditions are maintained by means described in the art and not directly addressed in this application. As the reaction proceeds, primers will extend and amplicons will accumulate on the beads. The success of PCR with forward primers attached to beads was demonstrated in Article [1] and related patents. Given a reasonable amplification efficiency, the total quantity of double-stranded DNA captured by the beads will approximately double with each cycle.

In embodiments of the methods, beads are attracted to an electromagnet at the end of the elongation step. (The measurement step need not be between the elongation step and the denaturation step, and the description is intended to be explanatory and not limiting.) Said electromagnet is hereinafter referred to as the hold coil. An electric current supplied through said coil (hereinafter, hold current) creates a magnetic field that polarizes the beads, endowing a bead with a magnetic dipole moment; a gradient of the field attracts the dipoles. The hold current may be dc or it may be another waveform. For typical superparamagnetic bead sizes known in the art and readily available for purchase, and at quantities of beads necessary to capture all amplicons, and for the geometric proportions of a sample space on the order of unity, and given a high enough magnetic field gradient, the attracted beads will form a suspension layer on the face of the sample space closest to the hold coil; the layer will be a monolayer; and the filling factor of said monolayer will be on the order of unity. Provided an appropriate hold current waveform, the beads will then settle within the layer, forming a bead-amplicon condensate.

The electrical conductivity and/or the complex permittivity of the settled condensate are then interrogated with one or two coils; a coil acts as an excitation source, a sensor, or both and the hold coil may serve as one or both. The sample space is placed in the electromagnetic field of an excitation coil consisting of one or more turns of a wire, conductor, or circuit board trace where said coil is not in direct contact with the reaction mix. Changes in said electrical properties of the condensate are synchronously measured with the help of a pickup coil that is not in direct contact with the reaction mix.

The excitation coil is driven at ac, that is, with a sinusoidal voltage at some frequency. The detection electronics monitor one or more ac signals, such as the voltage across, and/or the current through, the excitation and the pickup coils. In an embodiment, the amplitude of one of these ac signals is forced to stay constant by continuously adjusting the amplitude of the driving voltage. The monitored signals are phase-synchronously demodulated against the applied voltage: that is, the signal constituent at the driving frequency is isolated; its in-phase and out-of-phase components, relative to the phase of the excitation signal, are determined; the overall amplitude of the constituent is determined; and the phase difference between the constituent and the excitation signal is determined. The four quantities: the in-phase and out-of-phase amplitudes; the overall amplitude; and the phase of each monitored signal, hereinafter referred to as signal parameters, are then recorded.

In some embodiments, the excitation coil is driven at resonance. Said resonance exists because of the tank circuit formed by the coil's inductance and capacitances: either deliberately introduced, parasitic, inherent to the suspension layer, or a combination thereof. In these embodiments, the driving frequency is continuously adjusted so as to maintain resonance, and the frequency is recorded along with the determined parameters of the monitored signals. In some embodiments, the frequency is adjusted and recorded identically so as to maintain constant, or at a maximum, or at a minimum, the amplitude of one of the monitored signals.

The new wireless detection method disclosed herein additional to the detection method of Patent Application [10] employs the concept of a magnetic amplifier, or saturable reactor. The superparamagnetic beads serve as the magnetically saturable medium. The excitation coil, which in this method is the same as the hold coil, is driven at an ac frequency; the driving waveform is superimposed on the hold waveform. The magnetizing field of the coil saturates the beads during a portion of the ac period. The bead condensate is in the proximity of the pickup coil and its differential effective permeability affects the effective lumped inductance of the pickup coil. When the beads are saturated, the inductance is smaller than when they are not.

A sinusoidal voltage at a second frequency is applied to the pickup coil where said second frequency is such that the coil is at or near resonance when the beads are saturated. Said resonance exists because of the tank circuit formed by the coil's inductance and capacitances: either deliberately introduced, parasitic, inherent to the suspension layer, or a combination thereof. The amplitude of the second-frequency current through the pickup coil (or, equivalently, the effective lumped inductance of the pickup coil) will oscillate at the first frequency, and the phase shift of said amplitude with respect to the first applied voltage will depend on the electrical properties of the bead-amplicon condensate as elaborated in the following section. Within this method, the first-frequency amplitude and the phase of the second-frequency amplitude are the recorded parameters. In order to determine these parameters, the current through the pickup coil is first demodulated against the second frequency, then the resulting time-varying amplitude is demodulated against the first frequency.

In all embodiments of the methods, beads are redispersed within the mix following the recording of the parameters. The overall concentration of double-stranded DNA in the monolayer of beads is on the order 10⁻² by weight as calculated in the following section. This value is far above PCR-limiting DNA concentration [4]. Redistributing the beads as uniformly as possible within the reaction mix is desired for optimum efficiency of the subsequent amplification cycles. The dispersion is achieved by removing the hold magnetic field and by pulling the beads away from the hold coil and towards a separate electromagnet, the release coil. Coordinated electric current waveforms are supplied to the hold coil and the release coil in order to create magnetic fields that achieve said effect. Because the release coil is on the side of the sample space opposite to the region in which the condensate is formed during the measurement step, the release coil is not well suited for measurement purposes and therefore is not combined with the excitation coil or the pickup coil in the device disclosed herein. Following the redispersion or as the redispersion is underway, the next amplification cycle commences with the denaturation step.

In all embodiments of the methods, a change in one or more parameters of the bead-amplicon condensate, or in a predefined mathematical function thereof, is calculated for a given reaction cycle. For example, said function may be the ratio of the in-phase component of a parameter to the out-of-phase component of the same parameter, which is also the tangent of the phase angle of said parameter. This change is relative to the value of the parameter, or to the value of its function, for a predetermined cycle number. In the example, the difference may be taken to be between the value of the tangent measured for a given cycle and its value for the second cycle. The value of the mathematical function may be output and displayed in real time on a monitor, forming a kinetic amplification curve where the cycle number is on one axis and the value of the function, on the other.

Said change is compared to a predetermined threshold. When the change exceeds the threshold, or shortly thereafter, the reaction is terminated. The starting quantity of the template in the sample is decided from the number of reaction cycles required for the change to reach said threshold. Said cycle number need not be an integer, and is interpolated by fitting discrete cycle-by-cycle values. The larger the starting quantity of the template, the faster the accumulation of amplicons and the smaller said threshold cycle number is.

Real-time detection is an aspect of the invention; endpoint detection is not claimed herein. Combined with a similar (but contact) electrical detection method [8], endpoint detection reduces the dynamic range of the quantitation compared to a real-time technique.

Mechanism of Ation

The electrical conductivity of a mix containing dNTPs and DNA decreases as the nucleotides are incorporated into DNA, and conversely increases as DNA is digested into its constituent nucleotides [5]. Single nucleotides are freer to move in a fluid than a polymer chain; also, a DNA molecule is surrounded by counterions which are pulled from the solution, thereby decreasing the overall ionic strength of the mix. However the DNA concentration at which the effect is discernible is quite high. The digestion measurements in Article [5] were made at a DNA concentration 1.5 orders of magnitude higher than the PCR-limiting value of 10 ng/μl.

From a complementary angle, more DNA of a certain fixed length in a solution that does not also contain dNTPs yields more conductivity [3, 5, 8, 9]. There is no contradiction because in the latter case, more material and therefore more ions are added whereas in the former, matter is chemically transformed from one molecular state to another. More DNA also yields a higher dielectric constant [3], which is the relative real permittivity. The latter effect becomes dramatic at DNA concentrations exceeding the PCR-saturating limit by several orders of magnitude.

Concentrating PCR amplicons with the use of beads offers a sensitive means for quantitative detection of the amplification [8, 9]. In this art, endpoint detection was performed upon bead-bound amplicons extracted from a PCR mix and suspended in distilled water. Conversely, Article [5] measured the conductivity of amplicons (but not beads) dispersed in a PCR mix complete with salts and unused dNTPs and primers. The prophetic leap of faith herein stipulates that the extraction technique of Article [8] will yield a sufficiently sensitive and sufficiently specific outcome in a reaction mix when combined with wireless, electrodeless detection.

Indeed, the condensed bead monolayer has a thickness on the order of a handful of microns—the diameter of a bead. Given a sufficient density of binding sites, close to all of the amplicons will be contained within this layer. The volume of a typical PCR sample space is high tens to low hundreds of microliters, so the characteristic dimension of said space is mid- to high single millimeters. The concentration factor is therefore low to mid-thousands. The maximum concentration of amplicons achievable during the measurement step of the disclosed methods is three orders of magnitude higher than the PCR-limiting concentration and is larger than 10⁻² by weight.

Moreover, while the amplicons are isolated within the layer, unreacted dNTPs and unextended, unattached primers will remain floating in the mix so the appropriate comparison, for the purposes of detection contrast, is with the more-DNA studies referenced in the second paragraph of this section and not with the work converting DNA into dNTP mentioned in the first one. (Before any amplicons are extended on the beads, the attached forward primers constitute an initial quantity of bound DNA. The length of a primer is at least about one order of magnitude less than that of an amplicon and the latter is double-stranded during the measurement step whereas the primer is single-stranded. The detection contrast of the method therefore corresponds to an at least 20:1 ratio of the final to the initial concentration of DNA.)

Finally, the methods disclosed herein yield highly specific detection of a target DNA sequence over all other DNA that may be present in the sample because the latter DNA will not amplify and will not attach to the beads, remaining dispersed in the mix during the measurement step. Its concentration, relative to the target DNA, in the condensate layer is therefore reduced at least thousandfold. Nucleic acids in the condensate layer that are not attached to beads will still be detected but the background they present will be greatly reduced.

The mechanism of action of the underlying wireless detection method, in particular the mechanism tying bulk electrical properties of a reaction mix to the monitored signal(s), has been amply described in Patent Application [10]. In the present invention it is the electrical properties of a thin layer and not of the bulk of the sample space that are principally being probed; the key mechanisms, namely the interplay between, and contributions from, a magnetically induced eddy current and resistive shorting of turn-to-fluid pickup-coil capacitances, still underlie all three method embodiments disclosed herein. These methods mainly probe the electrical properties of the bead-amplicon condensate layer and not of the bulk reaction mix because the pickup coil is situated in the immediate proximity of the layer and because the effects produced by both mechanisms, and by the resistive-shorting phenomenon in particular, decrease significantly as the distance between the fluid region of interest and the pickup coil is increased. Elaborated upon in the remainder of the section is the particular new method of wireless detection involving a magnetically saturable reactor wherein the superparamagnetic beads are the saturating medium.

The effectiveness of using a saturable reactor is driven by the suppression of measurement errors produced by undesired coupling between the excitation coil and the pickup coil. In two-coil embodiments of the devices using the wireless detection method and disclosed in Patent Application [10], the dominating signal in the pickup coil is the directly induced voltage and not the voltage induced through the conductive fluid if the coils are helical or planar and not toroidal. The pickup coil and the excitation coil form a transformer and the pickup coil receives a large signal in the absence of any fluid medium in the sample space.

If the directly coupled signal is stable, it is a feature and not a bug. As explained in Patent Application [10], in these coil geometries the signal induced in the pickup coil owing to the conductivity and the permittivity of the reaction mix is several orders of magnitude smaller than the directly coupled signal; if the contribution from the conductivity dominates over that from the permittivity at a given frequency, the former signal is out of phase with the latter. Phase-synchronous detection allows for the calculation of the out-of-phase component with high precision even in the presence of a large in-phase contribution.

However, changes to the directly coupled signal owing to mechanically and thermally modulated changes in capacitive, as opposed to inductive, coupling can have an out-of-phase component and will then be commingled with the changes stemming from the varying conductivity of the fluid. The saturable-reactor concept introduces a frequency-mixer element that can separate, in frequency, the effects of inductive coupling between the coils, both direct and through the fluid, from those of capacitive coupling.

Indeed, the electric current through the pickup coil that is capacitively coupled from the excitation coil will be at the excitation frequency. If the pickup coil is separately driven at a higher frequency, demodulation and phase-sensitive detection at this pickup frequency will eliminate the capacitive contribution as well as the larger excitation-frequency component from direct inductive coupling between the coils. The current through the pickup coil will still bear a large contribution from direct inductive coupling but at the pickup frequency, not at the excitation frequency. Additional to this contribution, there will be a smaller term at the pickup frequency from coupling through the fluid. The amplitudes of the two terms oscillate at the excitation frequency and are out of phase. The smaller term is therefore unsullied by contributions of potentially similar or higher magnitude whose origin is not in the properties of the fluid.

The saturable-reactor method measures the frequency-dependent electrical conductivity and complex permittivity of a fluid at the excitation frequency and not at the pickup frequency. (To be precise, the foregoing discussion detailed the measurement of the conductivity and not of the permittivity. The measurement of the latter, through Kramers—Kronig relations, will be detailed in a subsequent application.)

Detailed Description of the Device

Four distinct device embodiments are described in the attached claims. Note that the arrangements and their description are not restricted to the particular superposition of the method embodiment, the binding chemistry, the sample space geometry, the device kind, and their mutual positioning, and the invention is intended to be limited only by the scope and the spirit of the appended claims.

The functionalized superparamagnetic beads that are an aspect of the invention are illustrated in FIG. 3 and are known in the art. A layer 304 of a binding moiety coats a bead 303 having a superparamagnetic core. In particular embodiments of the device, said layer is streptavidin, Tamavidin® 2, or Tamavidin® 2-HOT. Forward primers 305 and amplicons 306 attach to the bead 303 using a complementary moiety 307, such as biotin. Said attachment may be prior to the beginning of the chain reaction, or alternatively a reaction mix 301 may be introduced to the beads 303 that is a product of a number of cycles of a prior PCR. Said amplicons 306 are double-stranded at the end of the elongation step of PCR and become denatured during a subsequent step.

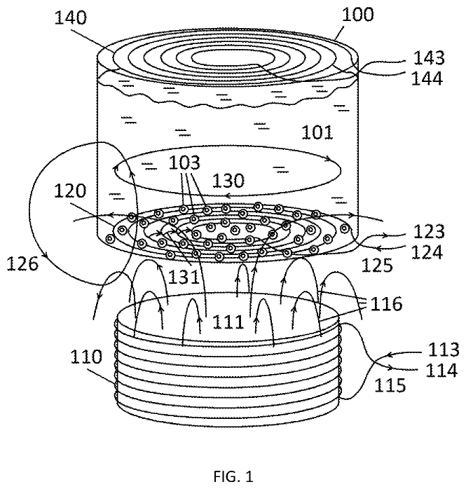

A device embodiment comprising three coils situated in the proximity of a sample well is illustrated in FIG. 1. Note that the arrangement and its description are not restricted to a cylindrical well geometry, and the invention is intended to be limited only by the scope of the appended claims. During the measurement step of the disclosed methods, an electric current 115 is supplied to a hold coil 110 that abuts the sample well 100 via terminals 113 and 114. Said current creates a magnetic field 116 whose gradient attracts superparamagnetic beads 103 to the hold coil 110 from within reaction mix 101. The magnitude of said field 116, and therefore of its gradient, are increased over hundredfold by the presence of a magnetically permeable core 111 compared to the case in which the coil 110 is not wound about a core composed of a material having a high magnetic permeability. The excitation coil 120 and the pickup coil 120 are one and the same planar coil. Said coil 120 is disposed in the immediate proximity of the reaction mix 101 and of the condensate layer comprising the beads 103 and attached primers and amplicons, said layer formed as a result of the attraction of the hold coil 110.

Said bead-amplicon condensate layer is interrogated by the coil 120. An ac voltage applied via terminals 123 and 124 drives an ac current 125 through the coil 120. The amplitude and the phase of the current 125, relative to the applied voltage, depend on the effective lumped impedance of the coil 120. The impedance is altered by the accumulation of the amplicons attached to the beads 103 in the layer of the mix 101 that is in the immediate proximity of the coil 120. Changes in the conductivity of the layer affect the lumped inductance through the back action of eddy current 130; this current is induced in the layer by the time derivative of the magnetic field 126 created by the current 125. Changes in the conductivity also affect the lumped capacitance and the equivalent parallel resistance of the coil 120, both via the eddy-current mechanism and by virtue of resistive shorting of the turn-to-fluid capacitances of the coil. Ionic and displacement currents 131 associated with the latter effect are particularly shown. The effective impedance is measured by dividing the applied voltage by the current 125 through the terminals 123 and 124. In an embodiment of the methods, the coil 120 is driven at resonance and the excitation frequency is continuously adjusted using the measured amplitude of the current 125 so as to minimize said amplitude. Said resonance exists because the circuit comprising said coil 120 is a tank circuit by virtue of comprising a capacitor. Parasitic and turn-to-fluid capacitances are an inherent part of the coil; these contributions may be supplemented by a capacitor externally provided as part of the measurement circuit connected to the coil 120.

Upon completion of the measurement step, the beads 103 are released from the vicinity of the hold coil 110 and from the proximity of the excitation and pickup coil 120 by terminating the hold current 115 and instead supplying an electric current through terminals 143 and 144 of a planar release coil 140. Said coil 140 abuts the sample well 100 and is in the vicinity of the reaction mix 101. The release coil 140 may instead be a helical coil or a coil of another geometry, and the invention is only intended to be limited by the scope of the attached claims.

In the second device embodiment, the role of the excitation coil is instead played by the helical coil 110. A separate drawing to illustrate said embodiment is not provided and the description of the embodiment instead refers to FIG. 1. The helical hold coil 110 attracts the beads 103 into a condensate layer as described. Said coil 110 also serves as the excitation coil and the planar coil 120, disposed in the immediate proximity of the reaction mix 101 and of the condensate layer, is the pickup coil. An ac voltage applied via the terminals 113 and 114 drives an ac current through the coil 110 that is additional to the hold current, and the two contributions constitute the total current 115 through the coil 110. The ac component of the current 115 creates an ac component of the total magnetic field 116. The time derivative of the ac component of the field 116 in turn induces ionic and displacement eddy current 130 in the condensate layer. (Because of the separation between the excitation coil 110 and the reaction mix 101, the prevailing mechanism of action is by the eddy current 130 and not via the resistive shorting of the turn-to-fluid capacitances.) The time derivative of the secondary magnetic field (not shown) created by the eddy current 130 induces an emf in the pickup coil 120. The emf is detected by monitoring the voltage across the terminals 123 and 124. The ac current through the excitation coil 110 may be additionally monitored if the resistive-shorting effects upon the coil 110 are comparable with those of the eddy current 130 on the same coil 110. Following the measurement step, the beads 103 are redispersed as described.

The third device embodiment employs the saturable-reactor detection method. A separate drawing to illustrate said embodiment is not provided and the description of the embodiment instead refers to FIG. 1. The helical hold coil 110 attracts the beads 103 into a condensate layer as described. Said coil 110 also serves as the excitation coil and the planar coil 120, disposed in the immediate proximity of the reaction mix 101 and of the condensate layer, is the pickup coil. An ac voltage at a first frequency applied via the terminals 113 and 114 drives an ac current at said first frequency through the coil 110 that is additional to the hold current, and the two contributions add to the total current 115 through the coil 110. The ac component of the current 115 creates an ac component at the first frequency of the total magnetic field 116 created by the coil 110. The magnitude of the voltage applied to the coil 110 is such that the magnetizing field corresponding to the total magnetic field 116 is sufficient, during a portion of a period of the first frequency, to cause magnetic saturation in the beads 103.

The time derivative of the ac component of the field 116 in turn induces ionic and displacement eddy current 130 at the first frequency in the condensate layer. (Resistive shorting of turn-to-fluid capacitances is not a material contributor to the mechanism of action of this device embodiment and the corresponding detection method.) Said eddy current 130 creates a secondary magnetic field (not shown) at the first frequency that is approximately 90° out of phase relative to the ac component of the field 116. The total magnetic field, which is the sum of the field 116 and the smaller secondary field, therefore exhibits a small lag relative to the phase of the field 116. The saturation effect in the beads 103 is accordingly phase-shifted relative to the ac component of the current 115, and the magnitude of said shift depends on the electrical conductivity of the suspension layer. The time dependence of the effective lumped inductance of the pickup coil 120 exhibits the same shift relative to the current 115 because said coil 120 comprises the beads 103 as its effective magnetic core.

The effective lumped inductance of the coil 120 is measured by applying a voltage at a second ac frequency across the terminals 123 and 124. Said second frequency is such that the coil 120 is at or near resonance during a portion of a period of the first frequency; driving the coil 120 to resonance increases the amplitude of the subsequently demodulated signal. Said resonance exists because the circuit comprising said coil 120 is a tank circuit as described.

The ac current 125 through the pickup coil 120 is demodulated against the voltage applied to the coil 120. The amplitude of the current 125 is inversely proportional to the absolute value of the equivalent impedance of the coil 120; said impedance primarily depends on the effective lumped inductance of the coil. The amplitude of the current 125 is then again demodulated but against the first frequency, and the phase shift of said amplitude serves to determine the conductivity of the bead-amplicon layer. Following the measurement step, the beads 103 are redispersed as described.

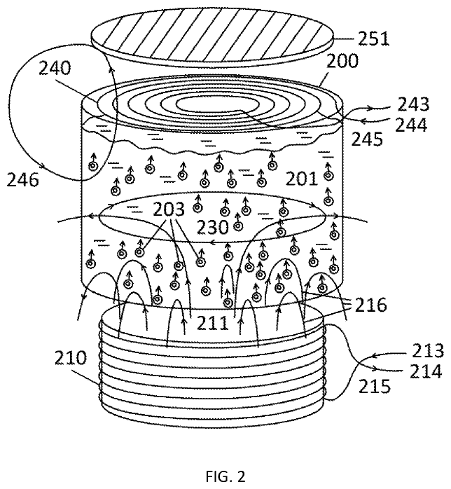

The fourth device embodiment combines the hold coil, the excitation coil, and the pickup coil into a single coil and is shown in FIG. 2. Illustrated additionally in the drawing is the behavior of the beads during the redispersion step of the disclosed methods.

The helical hold coil 210 wound about a magnetically permeable core 211 is disposed in the immediate proximity of sample well 200 and of reaction mix 201. The coil 210 attracts beads 203 into a condensate layer as described. Said coil 210 is the excitation coil and said coil 210 is the pickup coil. The coil 210 interrogates the bead-amplicon condensate. An ac voltage applied via the terminals 213 and 214 drives an ac current through the coil 210 that is additional to the hold current, and the two contributions constitute the total current 215 through the coil 210. The amplitude and the phase of the ac component of the current 215, relative to the applied ac voltage, depend on the effective lumped impedance of the coil 210. The impedance is altered by the accumulation of the amplicons attached to the beads 203 in the layer of the mix 201 that is in the immediate proximity of the coil 210.

Changes in the electrical conductivity of the layer affect the lumped inductance through the back action of eddy current 230; this current is induced in the layer by the time derivative of the magnetic field 216 created by the current 215. (Resistive shorting of turn-to-fluid capacitances is not a material contributor to the mechanism of action of this device embodiment owing to the comparatively larger distance between the turns of the coil 210 and the reaction mix 201.) Changes in the conductivity also affect, via the eddy-current mechanism, the equivalent parallel resistance of the coil 210. The effective impedance of the coil 210 is measured by dividing the applied voltage by the ac component of the current 215 through the terminals 213 and 214. In an embodiment of the methods, the coil 210 is driven at resonance and the excitation frequency is continuously adjusted using the measured amplitude of the current 215 so as to minimize said amplitude. Said resonance exists because the measurement circuit comprising said coil 210 is a tank circuit by virtue of comprising an externally provided capacitor.

Upon completion of the measurement step, the beads 203 are released from the proximity of the coil 210 by terminating the current 215 and instead supplying an electric current 245 through terminals 243 and 244 of a planar release coil 240. Said coil 240 abuts the sample well 200 and is in the vicinity of the reaction mix 201. The current 245 creates a magnetic field 246 whose gradient attracts the superparamagnetic beads 203 out of the settled layer and away from the hold coil 210, and towards the release coil 240 and into the reaction mix 201. The field 246 can be further focused inside the sample well 200 and into the reaction mix 201 by providing, in the proximity of the release coil 240, a fluxmat 251 made of a material having a high magnetic permeability and a high volume resistivity. The beads 203 can be further agitated and mixed within the sample well 200 by alternatingly attracting them to the coil 210 and the coil 240. Said attraction is achieved by supplying coordinated waveforms of the electric currents 215 and 245.

Finally, utility of the disclosed invention derives from having a plurality of the described devices within a single instrument. For example, the initial number of polymerase chain reaction cycles can be performed in a separate space and without detection or quantitation. Said initial reaction may be a multiplex reaction that produces amplicons specific to two or more target sequences. The reagent mix for said cycles is prepared in a proportion such that these initial cycles exhaust the reverse primers but not the biotin-conjugated forward primers. The quantity of a forward primer specific to each target in the initial mix is sufficient to subsequently proceed with the full number of cycles required for real-time quantitation.

The resulting reaction mix is then introduced into two or more devices described herein, each holding identical beads and initially holding reverse primers specific to a particular target sequence. Each device will amplify and detect a quantity of the template specific to the reverse primer contained therein. The invention is intended to be limited only by the scope and the spirit of the appended claims, so where a single sample space is shown, its description equally applies to an array of similar or identical spaces within an instrument.

Discussion

The discussion concerns aspects of the invention not addressed elsewhere.

(a) Method for non-contact measurement of the conductivity of a fluid involving a saturable reactor: The applicant believes the method to be both novel and considerably less obvious than the remainder of the methods for wireless measurement of conductivity disclosed herein and than the method for wireless measurement of the conductivity and permittivity disclosed in Patent Application [10]. While the latter method may be non-obvious when applied to the detection of nucleic acid amplification, similar methods and devices utilizing such methods for the measurement of the conductivity and permittivity of other fluid analytes are widely known in the art.

Measurements of the conductivity of a fluid analyte involving electrodes and two-frequency modulation are known in the art in general, and in the specific art of detecting the presence and measuring the quantity of nucleic acid molecules [7]. Instead of magnetic saturation, the method of Patent [7] employs the motion of the amplicons relative to the magnetically actuated beads and the deformation of the amplicons as the frequency-mixing agent. The applicant is unaware in prior art of a method for electrodeless measurement of the conductivity of a fluid analyte involving two-frequency modulation and a saturable reactor, and intends to submit a patent application for said method in the immediate future.

(b) qPCR vs. other real-time nucleic acid amplification schemes: The methods disclosed herein are not necessary for real-time detection of LAMP because an insoluble molecule is generated in its course and the additional step of concentrating the amplicons is not needed. The electrical conductivity and/or the complex permittivity of the bulk of the reaction mix are sufficiently altered by the amplification.

As dNTPs are incorporated into amplicons during PCR, and absent an ionic probe, the overall ionic strength of the reaction mix is not affected to nearly the same degree as it is for LAMP. The amplicon extraction step therefore increases the overall sensitivity of PCR detection and decreases the lower limit of the quantitation. The incentive of using PCR and not LAMP lies principally in the ability to use fully debugged reagent mixes and in the ability to multiplex the targets.

(c) Cycle-by-cycle detection: It is not necessary to perform the step of extracting, detecting, and redispersing the amplicons with every cycle of PCR. Said measurement step can be performed upon every other cycle, or, for example, every fifth cycle, or every fifth cycle until there is a significant change in the value of the kinetic-curve function described in Section 10 and every single cycle thereafter. The methods as described can also resume the amplification after a number of chain reaction cycles have been performed in a different sample space, or within the described sample space; after said cycles have been performed using a different method of detection and using devices not herein disclosed, or without detection.

The description of the methods and their embodiments in said section is not intended to be exhaustive, and the invention is intended to be limited only by the scope and the spirit of the appended claims.

(d) Magnetic-field effects: The ac magnetic field is applied to the bead-amplicon condensate layer in order to induce an eddy current and, in some embodiments of the methods, to magnetically saturate the beads so as to implement a frequency mixer. It is not believed that the ac magnetic field will induce motion or deformation of the amplicons relative to the beads to which they are attached. However, each bead-amplicon complex will certainly be exposed to the gradient of said ac field and individual complexes may move on a submicron scale. In other words, the methods disclosed herein will work—and may perform best—if the beads are completely stationary, although some motion is unavoidable.

(e) Measurement step: Said step naturally follows the elongation step of PCR and precedes the denaturation step; however an alternate sequence may be productive and the invention is only limited by the appended claims. For a given cycle of PCR, the amplicon payload attached to a bead is at a maximum following the elongation step and so the detection efficiency may then be the most optimum.

(f) Gravity: The hold coil need not be at the bottom of the sample space. Given sufficiently large hold and release magnetic fields, and provided enough reaction mix within the space, the device will operate in any orientation relative to the Earth's gravitational field.

Effectiveness

The advantages of using wireless detection of qPCR over fluorescent detection and over emerging alternative detection methods have been documented in Patent Application [10]. The overall complexity and cost of an instrument utilizing wireless detection are expected to be considerably less than those of an instrument that uses fluorescent detection. Wireless detection is generally expected to be as susceptible as, or less susceptible, to the sample matrix and sample contaminants as fluorescent detection. In the particular case of an optically opaque matrix, wireless detection wins.

Electromagnetic detection as disclosed herein amends wireless detection with an amplicon-concentrating step. Said addition increases the sensitivity and the specificity of the underlying method. The former is increased because the conductivity of nucleic acid material is measured in the aggregate and not in bulk. The latter is improved because only the specific amplicons are extracted and measured.

Intended Use

Electromagnetic detection of qPCR can be used in most contexts of molecular and genomic detection and molecular diagnostics. It is particularly appropriate for tough sample matrices: applied to food safety and forensics, as contrasted with medical diagnostics and drinking water. For example, the methods are expected to perform well with feces, ground plant and animal matter, DNA-containing swabs, and dried and reconstituted blood. Owing to their specificity, the methods are a suitable answer to the challenge of quantifying a small number of copies of a pathogen in the presence of a large amount of background DNA.

The methods are also an excellent fit for point-of-care and point-of-need applications because the associated device occupies little extra space and add little extra weight in excess of what is required for the amplification reaction itself. The associated circuitry consumes little extra power compared to what is needed to maintain the thermal regime of PCR. The resulting instrument can therefore be small, rugged, inexpensive, and battery powered.

The same, however, can be said of an instrument utilizing most known methods of direct electrical detection. The described methods shine in their specificity and in the insensitivity to the matrix and the contaminants. The insensitivity may enable direct detection of PCR products in a challenging sample without the need for a separate nucleic acid purification step in the sample processing workflow. Instead and compared with current art, the insensitivity may help eliminate an enrichment, or incubation, step. Eliminating the steps also obviates the need for sample transfer and thereby for a trained operator.

Alternatively, the amplification and detection subsystem can integrate tightly with a purification step known in the art, where sample transfer occurs automatically and entirely within the small and lightweight instrument. The synergy allows for the creation of a hands-off apparatus suitable for use at a point of need, for example for testing fresh produce that arrives at a restaurant for harmful bacterial pathogens or for surveying an agricultural environment for locations where said pathogens persist. Among components of such point-of-need instrument, the device herein described is envisioned as the last stage in the quantitation workflow.

Ground plant or animal matter first undergoes nucleic acid extraction and purification. After a number of initial PCR cycles, the reaction mix is again purified to retain only the shorter amplicons and primers. The eluted amplicons and primers are then deposited in said last stage, where the amplification reaction runs to saturation and where the specific amplicons are quantified in real time using the electromagnetic detection method.

While point-of-need food safety may present a particularly appealing application for the method, there is sufficient utility within a pure research instrument, for example a portable 96-well plate reader. The lack of electrical connections to the wells enables the creation of a general-purpose portable instrument that is entirely orthodox in the required workflow, and considerably less expensive than a qPCR fluorescence plate reader. Reliable and robust research results produced with wireless detection will increase the general acceptance of electromagnetic methods, without which further proliferation of fluorescence-less techniques is not possible.

Terminology

The number in parentheses refers to the section in which the term is first used.

(2) SAMPLE: Contains liquid water, a quantity (which may be zero) of the nucleic acid template molecule under examination, a matrix, contaminants, plus reagents such as buffers.

(2) REAGENT MIX: Known to contain zero quantity of the nucleic acid template molecule under examination.

(2) PCR: Polymerase chain reaction. A scheme of nucleic acid amplification.

(2) SUPERPARAMAGNETIC: Exhibiting a form of magnetism which appears in small ferromagnetic or ferrimagnetic particles. In the absence of an external macroscopic magnetizing field, a superparamagnetic particle exhibits a zero magnetic dipole moment. The magnetic susceptibility of a superparamagnetic particle is considerably larger than that of a paramagnetic particle. The zero-field volume magnetic susceptibility of commonly used superparamagnetic particles is on the order of unity.

(2) AMPLICON: A strand of DNA or RNA that is the product and/or source of amplification. Amplification generates other products, for example ions and insoluble molecules.

(2) ELECTROMAGNET: A magnet in which the magnetic field is produced by an electric current.

(2) COIL: One or more turns of a wire, conductor, or circuit board trace. A continuous ring electrode without a gap is not a coil. In common English, a coil is not an electrode unless it is in direct contact with the object, substance, or region entered and exited by electricity.

(2) ELECTRODE: A conductor through which electricity enters or exits an object, substance, or region.

(2) WIRELESS: Relating to a transfer of energy or information between two or more locations in space without use of electrical conductors.

(2) QUANTITATION: Determining the starting quantity.

(2) ELECTROMAGNETIC FIELD: One or more of: the macroscopic electric field, the macroscopic electric displacement field, the macroscopic magnetic field, or the macroscopic magnetizing field.

(2) REACTION MIX: A composition including one or more reagents and the sample under examination.

(4) NUCLEIC ACID TEMPLATE: A nucleic acid strand that is copied to form a new strand.

(4) KINETIC CURVE: A plot wherein time is on the horizontal axis and a mathematical function of parameters recorded during the amplification is on the vertical axis. Parameters are defined below.

(5) SCHEME: Herein refers to a prescribed routine that includes the recipes for the kind and the quantity of the reagents and the thermal protocol. An amplification scheme does not mention the exact means of detection of the reaction products. A scheme can be supplemented by a detection method.

(5) qPCR: Polymerase chain reaction wherein the quantity of the amplification products is monitored in real time and the starting quantity of a nucleic acid template is determined from the time it takes the products to reach a threshold.

(5) SIGNAL: A raw electric current or voltage; the instantaneous value thereof, as contrasted with the amplitude and the relative phase.

(5) INTERCALATING: Reversibly including or inserting into a material with a layered structure.

(5) LAMP: Loop-mediated isothermal amplification.

(5) ELECTRICAL DETECTION: Detection where the means are entirely electrical. Includes electrochemical voltammetry, contact impedimetry, and contactless capacitively coupled conductivity detection.

(5) TARGET PATHOGEN: An organism whose DNA or RNA contains the target sequence.

(5) CONCENTRATION: Herein, the property and not the action.

(5) TARGET SEQUENCE: A segment of the nucleic acid template that is bound by a primer. The target sequence may be the entire nucleic acid template.

(6) CURRENT: Electric current.

(6) AT OR NEAR RESONANCE: At a frequency within f/(2·Q) of a resonant frequency f of a tank circuit having a quality factor Q.

(6) PARAMETER: Herein, the amplitude of a signal, its phase relative to a carrier, or the in-phase or the out-of-phase amplitude of the signal relative to the carrier; additionally, the carrier frequency.

(6) SAMPLE SPACE: Contains the reaction mix during the entirety of the amplification reaction.

(6) FLUXMAT: A thin layer made of a material that has a high magnetic permeability and a low electrical conductivity, for example a sheet of ferrite.

(7) MAGNETIC: Applied to an object and not to a field nor a property: Having a relative permeability materially different from unity, or, equivalently, a magnetic susceptibility considerably larger than zero.

(9) IONIC CURRENT: Electric current where charge carriers are ions in a fluid.

(13) IONIC PROBE: A molecular ion used to study the properties of an analyte.

Trademarks

DNeasy® is a registered trademark of QIAGEN N.V. Tamavidin® 2 and Tamavidin® 2-HOT are registered trademarks of Japan Tobacco Inc.

1. A method for electrodeless electromagnetic detection of polymerase chain reaction products, and for the determination of the starting quantity of a nucleic acid template present in a sample, comprising:
attaching a plurality of forward primers at their 5′ ends to a plurality of superparamagnetic beads;
contacting a fluid sample including a quantity of a nucleic acid template molecule with a reagent mix where one or both of: said reagent mix; and said fluid sample; include a polymerase, and where one or both of: said reagent mix; and said fluid sample; include the forward primers attached to the beads, and where one or both of: said reagent mix; and said fluid sample; include a plurality of reverse primers;
obtaining a reaction mix by situating said reagent mix and said sample within a sample space;
providing a first coil and a second coil each consisting of one or more turns of a wire, a conductor, or a circuit board trace wherein said coils are not in direct contact with said reaction mix;
providing a third means where said means is none, or a third coil disposed in the proximity of the reaction mix, said coil consisting of one or more turns of a wire, a conductor, or a circuit board trace and wherein said coil is not in direct contact with the reaction mix;
creating conditions such that a polymerase chain reaction generates a product if the sample contains the template;
during a cycle of said reaction, supplying an electric current to the first coil attracting the beads to the vicinity of the coil;
during a cycle of said reaction, additionally applying an ac voltage to one of: the first coil or the third coil;
during a cycle of said reaction, producing an ac electromagnetic field in the vicinity of said coil where said vicinity encompasses the attracted beads;
during a cycle of said reaction, monitoring one or more of: the ac current through the first coil; the ac voltage across the third coil; or the ac current through the third coil;
during a cycle of said reaction, demodulating the monitored signal or signals against the applied voltage;
during a cycle of said reaction, determining the in-phase and out-of-phase components, and the amplitude and the phase of the demodulated signal or signals;
during a cycle of said reaction, recording said in-phase and out-of-phase components, and said amplitude or amplitudes and phase or phases, for said cycle;
during a cycle of said reaction, adjusting the amplitude of the applied ac voltage so as to maintain a constant amplitude of none or one of the demodulated signals;
during a cycle of said reaction, recording the amplitude of the applied ac voltage for said cycle;
during a cycle of said reaction, redispersing the beads as uniformly as practically possible within the reaction mix upon completion of the recording by supplying coordinated electric current waveforms to the first coil and the second coil;
measuring the cycle number, which need not be an integer, that most appropriately corresponds to a change in one or more of: the recorded quantities; or a function of said recorded quantities;
exceeding a predetermined threshold;
deciding upon the starting quantity of the nucleic acid template according to said cycle number wherein if said threshold is not reached, the template is not detected.

2. The method of claim 1 further comprising:
during a cycle of the reaction, adjusting the frequency of the applied ac voltage so as to maintain the amplitude of one of the demodulated signals at one of: a maximum, a minimum, or a constant value; and
during a cycle of said reaction, recording said frequency for said cycle, prior to the step of redispersing the beads as uniformly as practically possible within the reaction mix upon completion of the recording by supplying coordinated electric current waveforms to the first coil and the second coil.

3. A method for electrodeless electromagnetic detection of polymerase chain reaction products, and for the determination of the starting quantity of a nucleic acid template present in a sample, comprising:
attaching a plurality of forward primers at their 5′ ends to a plurality of superparamagnetic beads;
contacting a fluid sample including a quantity of a nucleic acid template molecule with a reagent mix where one or both of: said reagent mix; and said fluid sample; include a polymerase, and where one or both of: said reagent mix; and said fluid sample; include the forward primers attached to the beads, and where one or both of: said reagent mix; and said fluid sample; include a plurality of reverse primers;
obtaining a reaction mix by situating said reagent mix and said sample within a sample space;
providing a first coil and a second coil each consisting of one or more turns of a wire, a conductor, or a circuit board trace wherein said coils are not in direct contact with said reaction mix;
providing a third means where said means is none, or a third coil disposed in the proximity of the reaction mix, said coil consisting of one or more turns of a wire, a conductor, or a circuit board trace and wherein said coil is not in direct contact with the reaction mix;
creating conditions such that a polymerase chain reaction generates a product if the sample contains the template;
during a cycle of said reaction, supplying an electric current to the first coil attracting the beads to the vicinity of the coil;
during a cycle of said reaction, additionally applying an ac voltage at a first frequency to the first coil;
during a cycle of said reaction, producing an ac electromagnetic field at the first frequency in the vicinity of said first coil where said vicinity encompasses the attracted beads and wherein the macroscopic magnetizing field constituent of said electromagnetic field is sufficient to cause magnetic saturation in said beads;
during a cycle of said reaction, additionally applying an ac voltage at a second frequency to the third coil, where said second frequency is larger than the first frequency;
during a cycle of said reaction, producing an ac electromagnetic field at the second frequency in the vicinity of said third coil where said vicinity encompasses the beads attracted to the first coil;
during a cycle of said reaction, monitoring the ac current at the second frequency through the third coil;
during a cycle of said reaction, demodulating the monitored current against the second frequency;
during a cycle of said reaction, determining the amplitude of the demodulated current;
during a cycle of said reaction, demodulating said amplitude against the first frequency;
during a cycle of said reaction, determining the in-phase and out-of-phase components, and the first-frequency amplitude and the phase of the demodulated second-frequency amplitude;
during a cycle of said reaction, recording said in-phase and out-of-phase components, and said amplitude and phase, for said cycle;
during a cycle of said reaction, redispersing the beads as uniformly as practically possible within the reaction mix upon completion of the recording by supplying coordinated electric current waveforms to the first coil and the second coil;
measuring the cycle number, which need not be an integer, that most appropriately corresponds to a change in one or more of: the recorded quantities; or a function of said recorded quantities;
exceeding a predetermined threshold;
deciding upon the starting quantity of the nucleic acid template according to said cycle number wherein if said threshold is not reached, the template is not detected.

4. The method of claim 1 further comprising the initial steps of:
coating the superparamagnetic beads with a first binding moiety;
preparing the fluid sample as a mix of products of a prior polymerase chain reaction wherein forward primers are conjugated with a complementary binding moiety at their 5′ ends; and
attaching amplicons of said prior reaction to said beads by contacting said product mix with said beads,
and wherein the nucleic acid template molecule is an amplicon of said prior polymerase chain reaction.

5. The method of claim 2 further comprising the initial steps of:
coating the superparamagnetic beads with a first binding moiety;
preparing the fluid sample as a mix of products of a prior polymerase chain reaction wherein forward primers are conjugated with a complementary binding moiety at their 5′ ends; and
attaching amplicons of said prior reaction to said beads by contacting said product mix with said beads,
and wherein the nucleic acid template molecule is an amplicon of said prior polymerase chain reaction.

6. The method of claim 3 further comprising the initial steps of:
coating the superparamagnetic beads with a first binding moiety;
preparing the fluid sample as a mix of products of a prior polymerase chain reaction wherein forward primers are conjugated with a complementary binding moiety at their 5′ ends; and
attaching amplicons of said prior reaction to said beads by contacting said product mix with said beads, and wherein the nucleic acid template molecule is an amplicon of said prior polymerase chain reaction.

7. The method of claim 4, wherein the first binding moiety is one of: streptavidin, Tamavidin® 2, or Tamavidin® 2-HOT and the complementary binding moiety is biotin.

8. The method of claim 5, wherein the first binding moiety is one of: streptavidin, Tamavidin® 2, or Tamavidin® 2-HOT and the complementary binding moiety is biotin.

9. The method of claim 6, wherein the first binding moiety is one of: streptavidin, Tamavidin® 2, or Tamavidin® 2-HOT and the complementary binding moiety is biotin.

10. A device for electrodeless electromagnetic detection of polymerase chain reaction products, and for the determination of the starting quantity of a nucleic acid template present in a sample, utilizing the method of claim 1 and wherein
the first coil is a helical coil and the third means is a flat coil and is a spiral coil;
said first coil is disposed about a core having a high magnetic permeability and outside the sample space; and
one or both of: the ac current through the first coil; and the ac voltage across the third coil; are monitored.

11. A device for electrodeless electromagnetic detection of polymerase chain reaction products, and for the determination of the starting quantity of a nucleic acid template present in a sample, utilizing the method of claim 2 and wherein
the first coil is a helical coil and the third means is a flat coil and is a spiral coil;
said first coil is disposed about a core having a high magnetic permeability and outside the sample space;
the ac current through the third coil is monitored; and
the frequency is such that the circuit including said third coil is at or near resonance.

12. A device for electrodeless electromagnetic detection of polymerase chain reaction products, and for the determination of the starting quantity of a nucleic acid template present in a sample, utilizing the method of claim 2 and wherein
the first coil is a helical coil and the third means is not present;
said first coil is disposed about a core having a high magnetic permeability and outside the sample space;
said first coil is disposed in the proximity of the reaction mix;
the ac current through the first coil is monitored; and
the frequency is such that the circuit including said first coil is at or near resonance.

13. A device for electrodeless electromagnetic detection of polymerase chain reaction products, and for the determination of the starting quantity of a nucleic acid template present in a sample, utilizing the method of claim 3 and wherein
the first coil is a helical coil and the third means is a flat coil and is a spiral coil;
said first coil is disposed about a core having a high magnetic permeability and outside the sample space; and
the second ac frequency is such that the circuit including said third coil is at or near resonance during a part of a period of the first ac frequency.

14. The device of claim 10, wherein the second coil is a flat coil and is a spiral coil and said coil is disposed in the proximity of the reaction mix.

15. The device of claim 11, wherein the second coil is a flat coil and is a spiral coil and said coil is disposed in the proximity of the reaction mix.

16. The device of claim 12, wherein the second coil is a flat coil and is a spiral coil and said coil is disposed in the proximity of the reaction mix.

17. The device of claim 13, wherein the second coil is a flat coil and is a spiral coil and said coil is disposed in the proximity of the reaction mix.

18. The device of claim 14 containing a layer made of a material having a high magnetic permeability and a low electrical conductivity.

19. The device of claim 15 containing a layer made of a material having a high magnetic permeability and a low electrical conductivity.

20. The device of claim 16 containing a layer made of a material having a high magnetic permeability and a low electrical conductivity.

21. The device of claim 17 containing a layer made of a material having a high magnetic permeability and a low electrical conductivity.

22. An instrument comprising a plurality of the devices of claim 18.

23. An instrument comprising a plurality of the devices of claim 19.

24. An instrument comprising a plurality of the devices of claim 20.

25. An instrument comprising a plurality of the devices of claim 21.
